(19) United States
(12) Patent Application Publication
(10) Pub. No.: US 2024/0345091 A1
(43) Pub. Date: Oct. 17, 2024

(54) IN SITU OPTICAL BIOSENSING SYSTEM AND METHOD FOR MONITORING SEROTYPES

(71) Applicant: The State of Israel, Ministry of Agriculture & Rural Development, Agriculture research Organization, Rishon Lezion (IL)

(72) Inventors: Giorgi SHTENBERG, Herzliya (IL); Nanda KUMAR, Shimoga, Karnataka (IN); Shlomo BLUM, Rishon Lezion (IL)

(21) Appl. No.: 18/682,950

(22) PCT Filed: Aug. 10, 2022
(86) PCT No.: PCT/IB2022/057456

§ 371 Date: Feb. 12, 2024

Related U.S. Application Data
(60) Provisional application No. 63/232,213, filed on Aug. 12, 2021.

(51) Int. Cl. G01N 33/573; B01L 3/00; C12Q 1/37; G01N 33/543

(52) U.S. Cl. CPC G01N 33/573; B01L 3/502715; C12Q 1/37; G01N 33/54386; B01L 2200/16; B01L 2300/0654; B01L 2300/0809

(57) ABSTRACT

A label-free optical biosensing system and method provide high sensitivity and specificity for in situ detection and activity estimation of serotypes, such as Botulinum Neurotoxins (BoNT). Pre-fabricated thin-film support structures are treated with a competitive immunoassay coupled to a biochemical cascade reaction, which provides optical signal amplification. When the thin-film support structures receive a target analyte and are exposed to polychromatic light, reaction products cause a change in average refractive index which appears in reflectivity spectra measured by an optical interferometer. Optical signal amplification enables a linear response for serotype concentrations of only a few picograms per millilitre, as well as a level-of-detection threshold of 5.0 picograms per millilitre or less. The specificity and selectivity of the method have been verified in studies using various combinations of different serotypes as a target analyte. Similarly, the serotype activity is estimated by an adjunct sensing platform.

### CROSS-REFERENCE TO RELATED APPLICATIONS

This application is related to and claims priority from commonly owned U.S. Provisional Patent Application No. 63/232,213, entitled “In Situ Optical Biosensor for Monitoring Botulinum Neurotoxins (BoNT) Using Optical Signal Amplification”, filed on Aug. 12, 2022, the disclosure of which is incorporated by reference in its entirety herein.

### TECHNICAL FIELD

The present invention relates to a system and method for detecting and estimating the activity of serotypes in a sample, using an in-situ porous optical biosensor with optical signal amplification.

### BACKGROUND OF THE INVENTION

Botulinum neurotoxins (BoNT) are known to cause the severe neurological disease of botulism. These neurotoxins are immunologically classified into seven serotypes (named A-G) and their specific typing is important for epidemiological reasons. Despite the availability of vaccines against BoNT, there are still many outbreaks of botulism worldwide. BoNT-C and BoNT-D (including their mosaic variants) account for botulism in farm animals. In veterinary applications, an in-situ method for rapidly detecting the presence of botulism is especially important, in order to limit the economic loss sustained due to animal deaths and the contamination of animal feed and animal produce.

A variety of methods have been developed for detecting the presence of cell phenotypes in a biological sample. Among these methods, cell- or DNA-based assays and Enzyme-Linked ImunoSorbent Assay (ELISA) have been suggested as candidates for rapid, in situ monitoring. However, these techniques have been found to lack the sensitivity needed to satisfy BoNT limit of detection (LoD) requirements.

One relatively promising technique for in situ applications is the use of a porous Silicon (PSi) thin-film substrate and an optical biosensor based upon a Fabry-Pérot interferometer. For example, International Patent Publication number WO 2016/142878 A1 to E. Segal et al., entitled “Method of Determining Cellular Phenotypes”, published on Sep. 15, 2016, discloses methods of determining a phenotype of cells in a biological sample. The methods are based on measuring a refractive index of said cells based upon a diffraction pattern received from a diffraction grating having a plurality of compartments having lateral dimensions such that said cells can fit therein.

U.S. Patent Application Publication number US 2018/0100798 A1 to O. Du-Nour, entitled “High Sensitivity Real-Time Bacterial Monitor”, published on Apr. 12, 2018, teaches systems for the monitoring of bacterial levels in samples, using spectral analysis of pores having diameters enabling targets to enter them. The trapping pore array is cyclically illuminated by light of different wavelengths, and the light diffracted from the pore array is imaged by a two-dimensional detector array. Spectral analysis of the light intensity from this series of different wavelength enables the effective optical thickness (EOT) of each pore to be extracted.

### SUMMARY OF THE INVENTION

The present invention is directed to a label-free optical biosensing system and method having high sensitivity and specificity for in situ detection and activity estimation of serotypes. The system and method use a bio-functionalized, pre-fabricated thin-film support structure and an optical interferometer.

A competitive immunoassay coupled to a biochemical cascade reaction is used for optical signal amplification. The latter enables a linear response for serotype concentrations greater than a few picograms per millilitre (pg per mL), and a typical level of detection threshold of 5.0 pg per mL or less. The specificity and selectivity of the inventive method have been verified, in studies using various combinations of different serotypes as a target analyte.

According to one aspect of the presently disclosed subject matter, there is provided an optical biosensing system for in situ monitoring of serotypes. The system includes one or more pre-fabricated thin-film support structure(s) for receiving a target analyte, and also includes a bio-functionalizing reagent and/or an enzymatic amplification reagent for application to the support structure(s); a source of polychromatic illumination configured to illuminate the support structure(s) over a range of optical wavelengths; an optical interferometer configured to receive light scattered by the support structure(s) and to provide optical spectra over at least a portion of the range of optical wavelengths; a signal processor configured to analyze the optical spectra and to calculate output measurements of intensity, effective optical thickness (EOT), and incremental EOT values; and a biosensor monitor which receives the output measurements of the signal processor and calculates estimates of serotype detection probability and/or serotype concentration.

According to some aspects, the support structure(s) comprise a material selected from a group consisting of porous Silicon (PSi), alumina, platina, zinc oxide, and a polymer.

According to some aspects, the support structure(s) are cross-linked.

According to some aspects, the bio-functionalizing reagent includes a toxoid.

According to some aspects, the bio-functionalizing reagent includes an amino-modification agent.

According to some aspects, the enzymatic amplification reagent includes nanoparticles.

According to some aspects, wherein the range of optical wavelengths includes visual wavelengths and/or infrared wavelengths.

According to some aspects, the optical interferometer is a double layer, microcavity, Bragg reflector, or rugate interferometer.

According to some aspects, the signal processor is configured to implement a Reflective Interferometric Fourier Transform Spectroscopy algorithm and/or an Interferogram Average over Wavelength algorithm and/or a Morlet wavelet convolution algorithm.

According to some aspects, the serotypes include a Botulinum neurotoxin.

According to some aspects, a level of detection threshold of the serotypes is less than or equal to 5.0 picograms per millilitre.

According to another aspect of the presently disclosed subject matter, there is provided an optical biosensing method for monitoring serotypes, the method includes steps of fabrication of porous thin-film support structures for immunological recognition and proteolytic activity assays; bio-functionalization and attachment of a target analyte to the support structures; illumination of the support structures with polychromatic light and measurement of reflectance spectra over time; analysis of the spectra to determine intensity and effective optical thickness (EOT) values of serotype peaks in the spectra; calculation of optical relative activity and incremental EOT values of the serotype peaks; and calculation of estimates of serotype detection probabilities and concentrations.

According to some aspects, the bio-functionalization includes use of a toxoid and/or an amino-modification agent.

According to some aspects, the polychromatic light includes visual wavelengths and/or infrared wavelengths.

According to some aspects, the analysis includes use of a Reflective Interferometric Fourier Transform Spectroscopy algorithm and/or an Interferogram Average over Wavelength algorithm and/or a Morlet wavelet convolution algorithm.

According to some aspects, the calculation of estimates includes regression and activity status.

According to some aspects, the serotypes include a Botulinum neurotoxin.

### BRIEF DESCRIPTION OF THE DRAWINGS

Some embodiments of the present invention are herein described, by way of example only, with reference to the accompanying drawings. With specific reference to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1: An exemplary schematic of the optical biosensing system for in situ monitoring of serotypes, according to the invention.

FIGS. 2A and 2B: High resolution scanning electron microscope (HRSEM) micrographs of an exemplary porous silicon (PSi) thin film used in the system.

FIGS. 3A and 3B: Exemplary graphs of intensity versus EOT, after RIFTS processing.

FIGS. 4A and 4B: Exemplary experimental results for the BoNT-D specificity/selectivity and sensitivity of the system.

FIGS. 5A and 5B: Exemplary experimental results for the BoNT-C specificity/selectivity and sensitivity of the system.

FIGS. 6A-6G: Experimental results for the proteolytic activity of BoNT-C on a PSi film with SNAP25B/VAMP-2.

FIGS. 7A-7F: Experimental results for the analysis of real samples of BoNT-C and BoNT-D using both immunological recognition assays and proteolytic activity assays.

FIG. 8: An exemplary block diagram of the method for monitoring serotypes, according to the invention.

### DETAILED DESCRIPTION OF THE INVENTION

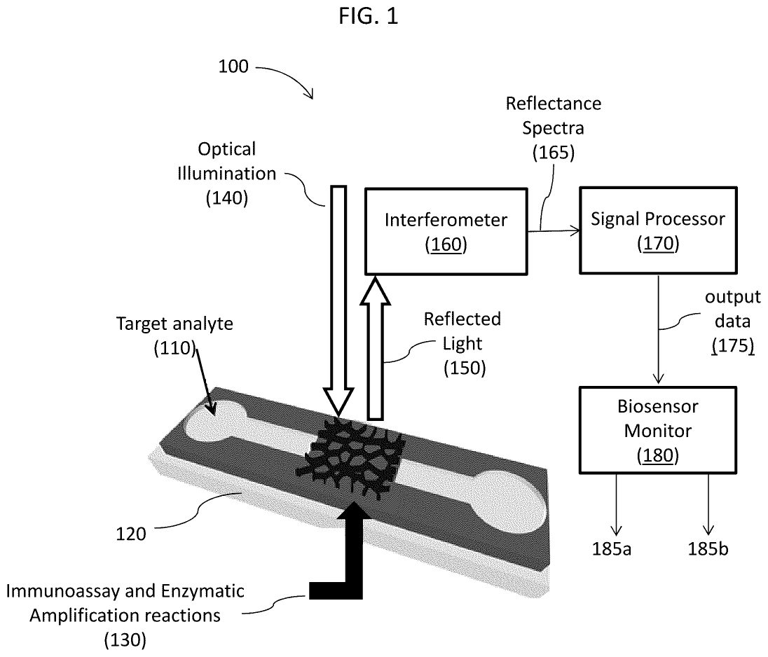

FIG. 1 shows a schematic of an exemplary optical biosensing system 100 for in situ monitoring of serotypes according to the invention. A target analyte 110 containing one or more BoNT serotypes is placed on a pre-fabricated thin-film support structure 120 having, for example, a porous Silicon (PSi) thin film. A competitive immunoassay with highly purified primary antibodies against the target analyte is applied, followed by an enzymatic amplification reaction 130. The latter uses horseradish peroxidase (HRP) pre-adsorbed secondary antibodies to oxidize the 4-chloro-1-naphthol (4CN) substrate and produce an insoluble precipitate which accumulates in the nanopores of the PSi film.

The reaction is monitored by exposing the PSi film to polychromatic, e.g. white light, optical illumination 140, and analyzing the reflected light 150 using a Fabry-Perot interferometer 160, over a wavelength range typically including both visible (VIS) and near-infrared (NIR) wavelengths. The presence of precipitate in the pores of the PSi film alters the refractive index and amplifies the intensity of the reflected optical signals. The interferometer 160 sends reflection spectra 165 to a real-time signal processor 170. The spectra are analyzed using Reflective Interferometric Fourier Transform Spectroscopy (RIFTS), Interferogram Average over Wavelength (IAW), and/or Morlet wavelet convolution algorithms. The details of the RIFTS and IAW algorithms are not recounted here, as these algorithms are familiar to those skilled in the art of PSi biosensing. The signal processor output data 175 includes, for example, a graph of intensity versus Effective Optical Thickness (EOT). The output data is sent to a biosensor monitor 180, which may be implemented, for example, in a general purpose digital computer. The monitor analyzes the output data 175 and generates estimates of detection probability 185a and concentration 185b for one or more BoNT serotypes.

The PSi film is fabricated by chemical and/or electrochemical anodization of a silicon wafer. By way of example, one such process involves the following steps. Two sequential electrochemical anodization steps are applied to a silicon wafer, such as a Boron-doped p-type Si wafer, having a resistivity of 1.0 milliohm-centimeter (mΩ·cm). The first anodization step is performed, for example, under a constant current density, e.g. 375 milliamps (mA) per square centimeter (cm²) for 30 sec. in 3:1 v/v ratio of aqueous 48% hydrofluoric (HF) acid and absolute ethanol (EtOH). The resulting layer is chemically detached by alkaline dissolution in 0.1 molar (M) sodium hydroxide, followed by a mild post-polishing treatment (1:3:1 v/v ratio of ultrapure water, EtOH and 48% HF acid, respectively) applied for 90 sec. each. The second anodization step includes, for example, a constant current density of 525 mA per cm² applied for 30 seconds. The freshly etched PSi thin films are thermally oxidized for example at 800° C. for 1 hour in a tubular furnace under ambient conditions producing oxidized nanostructures (PSiO₂). The oxidation process is a crucial passivation step in order to preserve the optical properties of the PSi film and to minimize surface-related aging effects in aqueous media. The above fabrication process is exemplary; other processes may be used by those skilled in the art in order to fabricate porous matrices of various physical dimensions while preserving the capabilities of the optical interferometer.

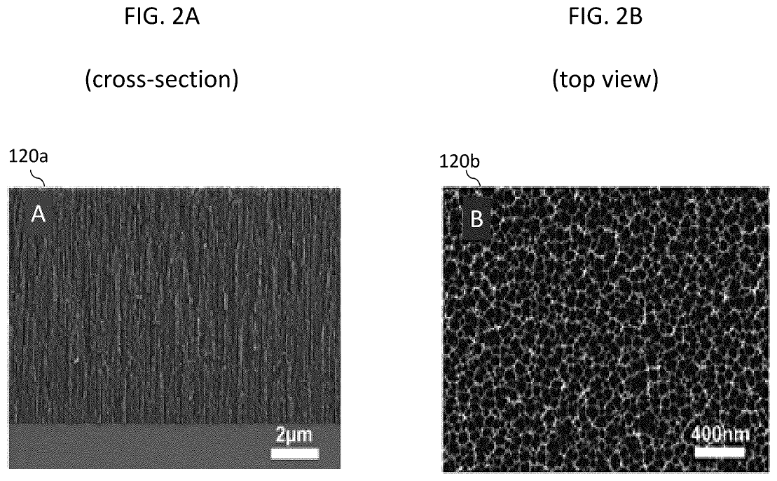

FIGS. 2A and 2B show high resolution scanning electron microscope (HRSEM) micrographs of an exemplary porous Si thin film used in system 100. FIG. 2A is a cross-sectional view of the thin film, in which the porous layer has a depth of about 8.5 microns (μm). FIG. 2B shows a top view of the PSi thin film at a high magnification. The pores in the silicon are roughly cylindrical, with diameters of 70±20 nm.

The PSi film then undergoes a bio-functionalization process. By way of example, one such process involves the following steps. The PSi nanostructures are physically adsorbed with 50 microliters (μL) of gelatin solution (10 mg mL⁻¹) applied for 30 min., followed by a HEPES (N-2-hydroxyethylpiperazine-N′-2-ethanesulfonic acid) buffer (pH 7.4) vigorous wash to remove loosely bound molecules. The resulting thin films are then cross-linked using glutaraldehyde (GluAld) solution (2.5% v/v). Immediately, 20 μL of BoNT-C toxoid (1 μg mL⁻¹) is applied on the GluAld modified surface for 30 min. at room temperature and allowed to incubate overnight at 4° C. Later, the PSi films are thoroughly rinsed with HEPES buffer (pH 7.4) to remove loosely bound molecules.

In some embodiments of the invention, bio-functionalization may involve steps such as: amino-modification using Aminopropyltriethoxysilane (APTES) and/or diisopropylethylamine (DIEA), anchoring Synaptobrevin/Vesicle-associated membrane protein (SNAP25B/VAMP-2) within the PSi nanostructure, and attaching zinc oxide nanoparticles (ZnO-NPs) to enhance the optical amplification.

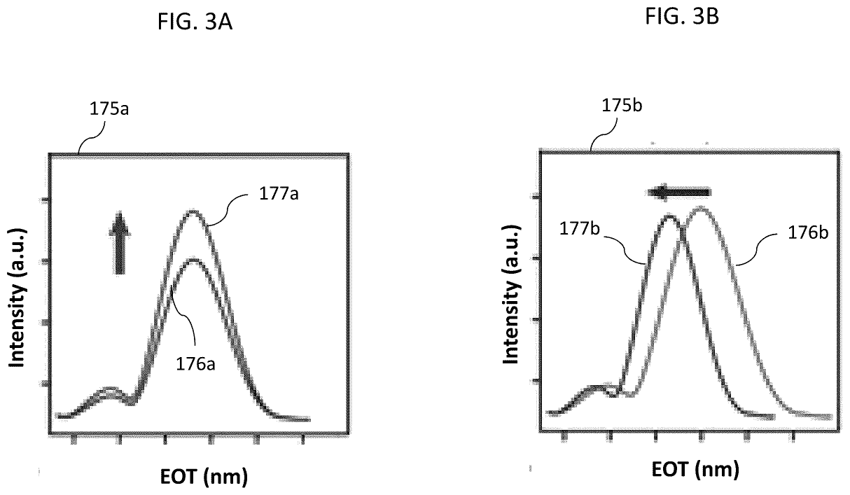

FIGS. 3A and 3B shows exemplary graphs, 175a and 175b, of intensity, in arbitrary units (a.u.), versus Effective Optical Thickness (EOT), in nanometers (nm), after RIFTS processing. The EOT is equal to two times the product of the average refractive index (n) of the porous layer and the total depth (L) determined by the Fabry-Pérot depth equation.

In FIG. 3A, the characteristic peaks 176a and 177a correlate with the infiltration of enzymatic reaction products into the pores of the PSi film, at two successive times after the start of the enzymatic reaction. The reaction products accumulate within the pores of the thin-film support structure, causing a change in average refractive index, which appears in the reflectivity spectra measured by the optical interferometer. The arrow indicates a shift to higher intensity, and thus higher optical amplification, as the reaction progresses.

In FIG. 3B, the characteristic peaks 176b and 177b correlate with the proteolytic reaction products which cause a shift in EOT, referred to as ΔEOT, at two successive times after the start of the proteolytic reaction. The arrow indicates the shift to lower values of EOT, as peptide fragments decrease due to specific cleavage.

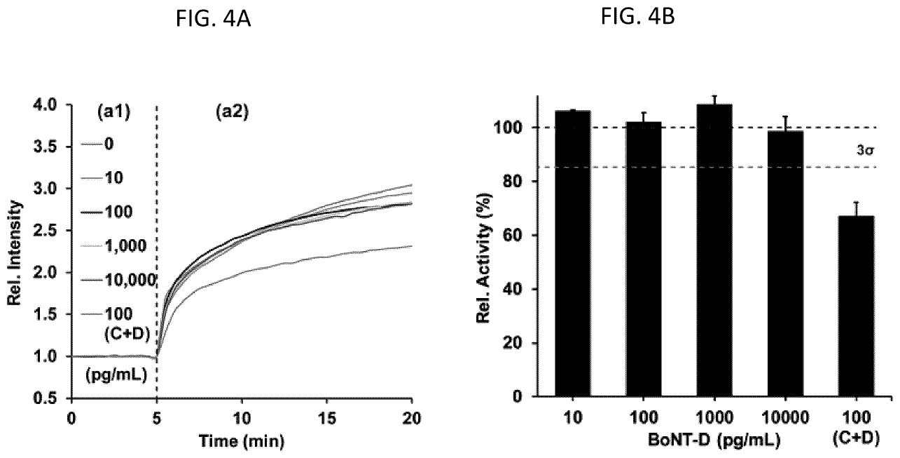

FIGS. 4A and 4B show exemplary graphs of experimental results for the BoNT-D specificity/selectivity and sensitivity, for system 100. The relative activity in percent is plotted on the vertical axis, for analytes containing different concentrations of BONT-D, and for a mixture containing of 100 μg per mL of BoNT-C and 100 μg per mL of BoNT-D.

The relative activity is computed as follows. First, a relative intensity of the observed peak in FIG. 3 is computed via:

$$Rel . Intensity = \frac{{A}_{BoNT}}{{A}_{0}} \qquad ( equation 1 )$$

where the numerator is a steady-state intensity after activation, which is typically reached about 20 minutes after HRP activation; and the denominator is a baseline intensity recorded prior to biochemical response activation.

Second, the relative activity is calculated for different BONT mixtures (and concentrations) via:

$$Rel . activity ( \% ) = \frac{Rel . {Intensity}_{BoNTx} - 1}{Rel . {Intensity}_{BoNT 0} - 1} \times 100 \qquad ( equation 2 )$$

In equation 2, the numerator is the relative intensity of the specific mixture, and the denominator is the maximal relative intensity without the addition of BoNT onto the optical platform.

In FIG. 4A, the relative intensity is plotted on the vertical axis versus reaction time in minutes. The thin film is fixed in a custom-made flow cell and the reflectivity spectra are recorded every 30 sec. The sensorgrams present an optical baseline with 0.8 mM 4CN in HEPES buffer (a1) followed by the addition of hydrogen peroxide (H₂O₂) to the cycled solution (a2). The biosensor is fixed in a custom-made flow cell and the reflectivity spectra are recorded every 30 sec. After about 20 minutes, the relative activities approach steady-state values.

In FIG. 4B, the relative activity data are reported as mean±one-sigma (standard deviation, with N=4). The horizontal red dashed line indicates the 3-sigma level used to calculate an LoD threshold. The results obtained are below the calculated LoD threshold, thus indicating a highly specific biosensing platform. Moreover, for the mixture containing of 100 μg per mL of BoNT-C and 100 μg per mL of BoNT-D, the relative activity decreases with respect to the positive control by 67±5%, thus indicating a highly selective biosensing platform.

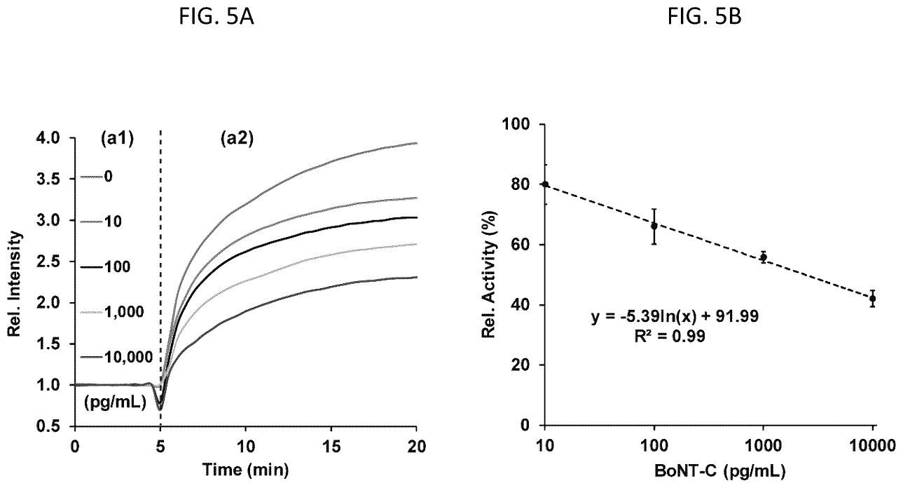

FIGS. 5A and 5B show exemplary experimental results for the BoNT-C specificity/selectivity and sensitivity for the in situ optical biosensor of the invention. In FIG. 5A, the relative intensity is plotted on the vertical axis versus reaction time in minutes, for different values of concentration ranging from 0 to 10,000 μg per mL. After about 20 minutes, the relative activities approach steady-state values. In FIG. 5B, the relative activity in percent is plotted on the vertical axis versus the toxoid concentration of BoNT-C on the horizontal axis, measured in units of pg per mL. Note that the horizontal axis is a logarithmic scale covering three orders of magnitude. The data are reported as mean±one-sigma (standard deviation, with N≥3). The dashed line represents an empirical linear relationship between relative activity and the logarithm of the concentration.

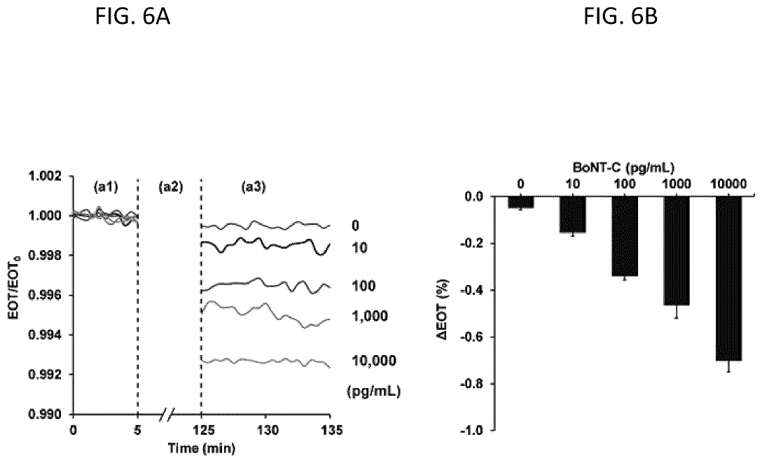

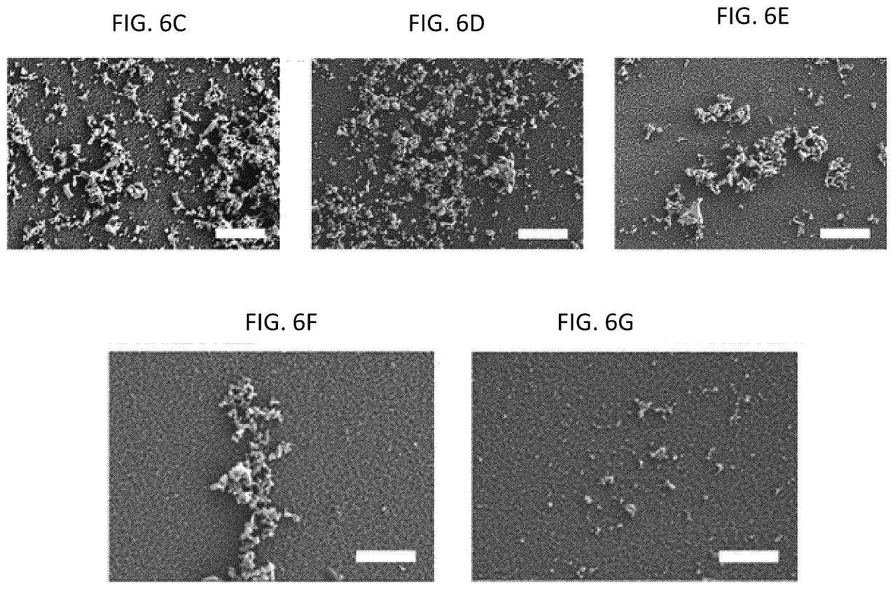

FIGS. 6A-6G present exemplary experimental results for the proteolytic activity of BONT-C on a PSi film with SNAP25B/VAMP-2. The sensorgrams in FIG. 6A present an optical baseline with HEPES buffer (a1), after which the toxoid, at various concentrations, is injected and allowed to interact for 120 min (a2), followed by buffer wash to remove any unbound proteolytic reaction fragments from the porous scaffold (a3). The biosensor is fixed in a custom-made flow cell and the reflectivity spectra are recorded every 30 sec. FIG. 6B shows the averaged net optical response of the BoNT-C toxoids. The data are reported as mean±standard deviation (N≥3). HRSEM micrographs following the RIFTS experiments are shown in FIGS. 6C-6G, corresponding to concentrations of 0, 10, 100, 1,000, and 10,000 μg per mL, respectively. The scale bar is 2 μm in each micrograph.

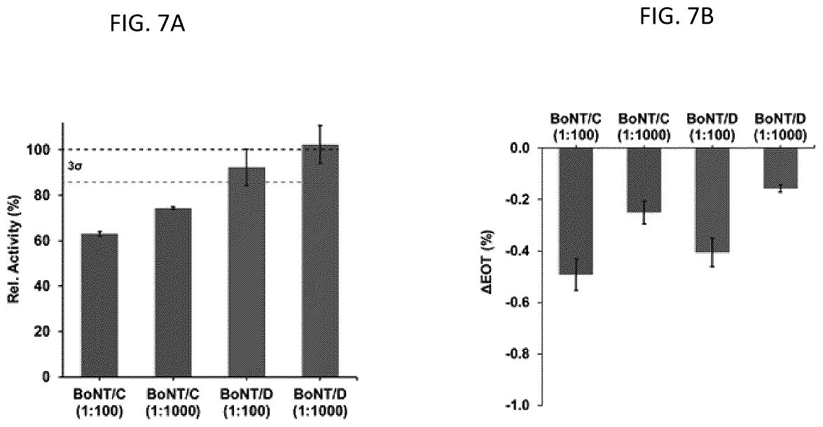

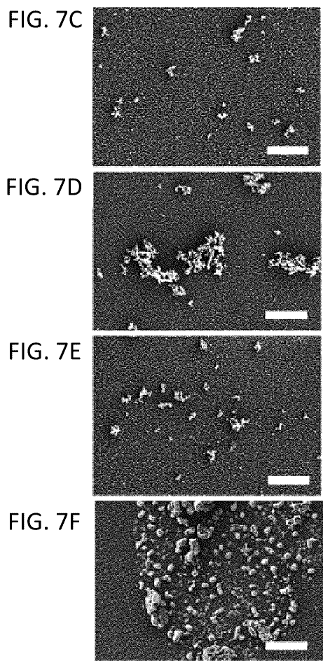

FIGS. 7A-7F present experimental results for the analysis of real samples of BoNT-C and BoNT-D using both immunological recognition assays and proteolytic activity assays. FIG. 7A shows graphs of the relative activity of the two toxins at two dilutions: 1:100 and 1:1000. The increase in relative activity at higher dilution indicates lower toxin content, resulting in augmented catalytic activities of the HRP cascade reactions. The horizontal red dashed line indicates the calculated LoD threshold based upon three standard deviations. For the BoNT-D samples, the measured relative activity is above the red dashed line, indicating that the developed assay toward BoNT-C is highly specific.

FIG. 7B shows the averaged net optical response of the corresponding proteolytic activities on PSi film modified with SNAP25B/VAMP-2. Data are reported as mean±one standard deviation (N=3). As the dilution increases from 1:100 to 1:1000, the ΔEOT values of BoNT-C change from −0.49±0.06% to −0.25±0.05%, and those of BoNT-D change from −0.41±0.06% to −0.16±0.01%.

FIGS. 7C-7F present HRSEM micrographs, corresponding to 1:100 BoNT-C, 1:1000 BONT-C, 1:100 BONT-D, and 1:1000 BONT-D respectively. The scale bar is 2 μm in each micrograph.

Table 1 below presents calculations of the BoNT-C and BoNT-D concentrations in real samples using measured relative activity values and the regression line for BoNT-C of FIG. 5B.

TABLE 1
Estimation of BoNT/C and BoNT/D toxins in real samples.

| | | Calculated BoNT | Proteolytic | |
|---|---|---|---|---|
| Serotype | Dilution | (pg mL⁻¹) | activity | Lethality |
| BoNT-C | 1:100 | 217 ± 26 | + | + |
| | 1:1000 | 26 ± 3 | + | − |
| BoNT-D | 1:100 | 4 ± 5 | + | N/A |
| | 1:1000 | 1 ± 1 | + | N/A |

Data are reported as mean ± standard deviation (N = 3).

The calculated BoNT-C concentration decreases by a factor of about 8.3 from 217±26 to 26±3 pg per mL for the 1:100 and 1:1000 dilutions, respectively. Furthermore, the calculated values of both BoNT-D samples are within or below the system's LoD (i.e., 4.2 pg per mL), indicating that the BoNT-D content in the real samples is insignificant. All of the samples in Table 1 demonstrate proteolytic activity.

Lastly, Mouse Laboratory Assay (MLA) studies were performed to confirm the lethality of the BoNT-C target analytes, by injecting BoNT/C samples into laboratory animals. The “Lethality” column in Table 1 indicates that the lethal 1:100 dilution is readily distinguished from the non-lethal 1:1000 dilution using the in-situ optical biosensor of the invention.

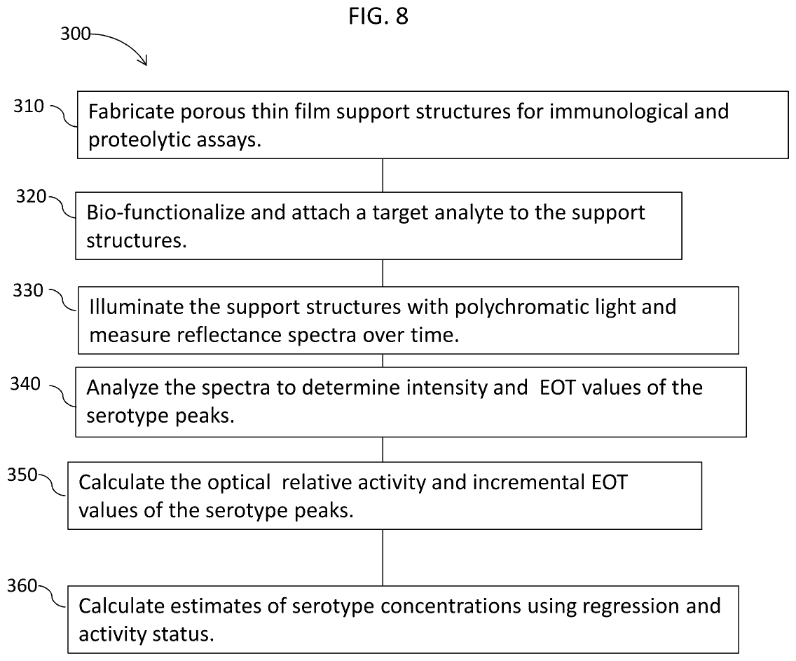

FIG. 8 shows an exemplary block diagram of the method 300 for monitoring of serotypes, according to the invention. The method consists of the following sequential steps:
Step 310: Fabrication of porous thin-film support structures for immunological recognition and proteolytic activity assays;
Step 320: Bio-functionalization and attachment of a target analyte to the support structures;
Step 330: Illumination of the support structures with polychromatic light and measurement of reflectance spectra over time;
Step 340: Analysis of the spectra to determine intensity and EOT values of serotype peaks in the spectra, using for example RIFTS and/or IAW;
Step 350: Calculation of the optical relative activity and ΔEOT values of the serotype peaks; and
Step 360: Calculation of estimates of serotype detection probabilities and concentrations using regression and activity status.

The descriptions of the various embodiments of the present disclosure have been presented for purposes of illustration but are not intended to be exhaustive or limited to the embodiments disclosed. For example, although the monitoring of BoNT-C and BoNT-D serotypes has been used to illustrate the use of the in situ optical biosensor of the invention, both the system and the method of the invention are readily applicable to all seven BoNT serotypes (including mosaic variants) and to other serotypes as well. Also, other porous and reflective nanostructures, e.g., Alumina, platina, zinc oxide, polymers, and others reflective platforms with distinct characteristic refractive indices, are within the scope of the invention. Other optical designs, using for example double layer, microcavity, Bragg reflector, rugate and other types of interferometers, are within the scope of the invention. Alternative surface modifications that attach a simulant molecule to the surface of a porous or solid support structure are also within the scope of the invention, as well as any type of nanoparticle, e.g. zinc oxide and gold, that can be used for signal amplification.

Many other modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. An optical biosensing system for in situ monitoring of serotypes, the system comprising one or more pre-fabricated thin-film support structure(s) for receiving a target analyte, and further comprising:
a bio-functionalizing reagent and/or an enzymatic amplification reagent for application to the support structure(s);
a source of polychromatic illumination configured to illuminate the support structure(s) over a range of optical wavelengths;
an optical interferometer configured to receive light scattered by the support structure(s) and to provide optical spectra over at least a portion of the range of optical wavelengths;
a signal processor configured to analyze the optical spectra and to calculate output measurements of intensity, effective optical thickness (EOT), and incremental EOT values; and
a biosensor monitor which receives the output measurements of the signal processor and calculates estimates of serotype detection probability and/or serotype concentration.

2. The system of claim 1 wherein the support structure(s) comprise a material selected from a group consisting of porous Silicon (PSi), alumina, platina, zinc oxide, and a polymer.

3. The system of claim 1 wherein the support structure(s) are cross-linked.

4. The system of claim 1 wherein the bio-functionalizing reagent comprises a toxoid.

5. The system of claim 1 wherein the bio-functionalizing reagent comprises an amino-modification agent.

6. The system of claim 1 wherein the enzymatic amplification reagent comprises nanoparticles.

7. The system of claim 1 wherein the range of optical wavelengths includes visual wavelengths and/or infrared wavelengths.

8. The system of claim 1 wherein the optical interferometer is a double layer, microcavity, Bragg reflector, or rugate interferometer.

9. The system of claim 1 wherein the signal processor is configured to implement a Reflective Interferometric Fourier Transform Spectroscopy algorithm and/or an Interferogram Average over Wavelength algorithm and/or a Morlet wavelet convolution algorithm.

10. The system of claim 1 wherein the serotypes comprise a Botulinum neurotoxin.

11. The system of claim 1 wherein a level of detection threshold of the serotypes is less than or equal to 5.0 picograms per millilitre.

12. An optical biosensing method for monitoring serotypes, the method comprising steps:
a) fabrication of porous thin-film support structures for immunological recognition and proteolytic activity assays;
b) bio-functionalization and attachment of a target analyte to the support structures;
c) illumination of the support structures with polychromatic light and measurement of reflectance spectra over time;
d) analysis of the spectra to determine intensity and effective optical thickness (EOT) values of serotype peaks in the spectra;
e) calculation of optical relative activity and incremental EOT values of the serotype peaks; and
f) calculation of estimates of serotype detection probabilities and concentrations.

13. The method of claim 12 wherein the bio-functionalization in step b) comprises use of a toxoid and/or an amino-modification agent.

14. The method of claim 12 wherein the polychromatic light in step c) comprises visual wavelengths and/or infrared wavelengths.

15. The method of claim 12 wherein the analysis of step d) comprises use of a Reflective Interferometric Fourier Transform Spectroscopy algorithm and/or an Interferogram Average over Wavelength algorithm and/or a Morlet wavelet convolution algorithm.

16. The method of claim 12 wherein the calculation of estimates in step f) comprises regression and activity status.

17. The method of claim 12 wherein the serotypes comprise a Botulinum neurotoxin.
